(19) United States
(12) Patent Application Publication
(10) Pub. No.: US 2013/0018336 A1
(43) Pub. Date: Jan. 17, 2013

(54) NOVEL DRESSING INCLUDING A WEB OF MICROFIBERS OR NANOFIBERS SUITABLE FOR GELLING OR SOLUBILIZING

(71) Applicant: Jean-Marc Pernot, Dijon (FR)

(72) Inventor: Jean-Marc Pernot, Dijon (FR)

(73) Assignee: LABORATOIRES URGO, Chenove (FR)

(21) Appl. No.: 13/521,991

(22) PCT Filed: Jan. 13, 2011
(86) PCT No.: PCT/FR11/50063

§ 371 Date: Oct. 1, 2012

(30) Foreign Application Priority Data
Jan. 14, 2010 (FR) 1050222

(51) Int. Cl. A61L 15/24; A61F 13/56; A61K 31/728; A61M 35/00; A61L 15/26; A61L 15/28

(52) U.S. Cl. 604/307; 424/445; 514/54; 604/365

(57) ABSTRACT

The present invention relates to a repositionable bandage including an adhesive hydrocolloid mass, wherein said bandage includes, on at least one portion of the surface of the adhesive mass intended for being in contract with the wound when the bandage is being used, a web of microfibers or nanofibers, at least 90% and preferably at least 95% of which being made up of one (or more) material(s) selected among natural or synthetic polymers, and being suitable for solubilizing or gelling in under ten seconds, and preferably still in less than one second when in contact with the exudate from the wound. The bandage according to the invention is intended for treating wounds such as blisters, exuding wounds, burns and dermal-epidermal lesions, whether superficial, deep, chronic or acute.

A subject matter of the present invention is a novel repositionable wound dressing comprising an adhesive hydrocolloid mass, which dressing is intended for the treatment of wounds, such as exudative wounds, burns, superficial or deep dermo-epidermal lesions, which may be chronic or acute, and in particular for the treatment of blisters.

Dressings comprising hydrocolloids have been known for more than 20 years. They are composed of a backing on which is deposited an adhesive mass comprising hydrocolloids. Mention may be made, as examples, of the products sold under the names Algoplaque® by Laboratoires URGO and Comfeel® by Coloplast. Dressings comprising an adhesive mass comprising hydrocolloids, specifically intended for the treatment of blisters, are also known and are sold, for example, under the names Urgo Traitement Ampoules® by Laboratoires URGO and Compeed® by Johnson & Johnson.

In order to make possible good absorption of the exudates from the wound, these dressings comprise relatively high amounts (of the order of 20 to 50% by weight) of hydrocolloids. Preferably, these dressings are designed to be held in place without the help of an additional adhesive tape, by adhering directly to the skin.

The adhesive mass of these known dressings is normally composed of a hydrophobic continuous phase in which is dispersed a noncontinuous phase of hydrocolloid particles intended to absorb the exudates from the wound.

The absorption of the exudates by the hydrocolloids causes the adhesive mass to gel, which makes it possible to painlessly remove the dressing from the wound after it has been used.

In order to provide for the maintenance over time of their absorption capacity and of their cohesion during removal, these dressings have a high initial adhesiveness. This is even truer for the dressings intended for the treatment of blisters, which have to be positioned in areas which are curved or difficult to access and which are subject to high mechanical stresses during their use.

In order to increase the adhesiveness of these dressings, work has been carried out:
on the one hand, on the qualitative and quantitative composition of the adhesive masses making up the dressings; and
on the other hand, at the shape of these dressings, in particular by giving them beveled edges.

Such dressings and their compositions are well known and are described, for example, in the documents EP 264 299, EP 503 029, EP 1 020 198 and FR 2 495 473.

However, due to their high adhesiveness, these dressings cannot be easily repositioned during their deployment on the skin of the patient. This is because the removal of these dressings is very painful as the dressing adheres to the wound or the blister, as long as it has not absorbed the exudates, which may require a relatively long time of the order of 15 minutes or more.

However, the repositioning of the dressing during its deployment is very often necessary, for example when the surface of the body on which this dressing has to be applied is not flat, and this problem is well known to nursing personnel.

In addition, these dressings are all the more difficult to deploy as they are generally very fine and, in the case of dressings intended for the treatment of blisters, small in size and as they have to be positioned in places which are difficult to access: toes, arches of the foot, heels, and the like.

Many users have already been confronted with this problem of repositioning, particularly in the case of dressings intended for the treatment of blisters, which it is also desirable to be able to reposition for reasons of unobtrusiveness and attractiveness, for example to avoid the formation of folds or to conceal the dressing behind the strap of a shoe.

Although numerous applicator systems have been developed for twenty years to make it easier to put on these dressings, these systems do not, however, make it possible to satisfactorily solve the problem of the repositioning and in particular there does not currently exist an ideal solution to the problem of the painless removal of an adhesive dressing immediately after a first deployment.

Under these conditions, it is an aim of the present invention to solve the novel technical problem consisting in the provision of an adhesive hydrocolloid dressing of a novel design, which can be painlessly removed immediately after it has been put on in order to be repositioned one or more times, while retaining its properties of adhesion, cohesion and absorption over time.

It has been discovered, and this constitutes the basis of the present invention, that it was possible to solve this technical problem, in a relatively simple way which can be used on an industrial scale, by modifying the surface intended to come into contact with the skin of a conventional hydrocolloid adhesive dressing by depositing thereon a web of microfibers or nanofibers capable of dissolving or gelling on contact with the exudates from the wound substantially completely and in a very short period of time, preferably of less than 10 seconds and more preferably of less than 1 second.

Thus, according to a first aspect, of the present invention concerns a wound dressing comprising an adhesive mass comprising hydrocolloids, characterized in that the dressing comprises, on a portion at least of the surface of the adhesive mass intended to come into contact with the wound in the position of use, a web of microfibers or nanofibers which are composed, for 90% at least and preferably 95% at least of them, of one (or more) material(s) chosen from natural or synthetic polymers, and capable of dissolving or gelling in less than 10 seconds and more preferably in less than 1 second on contact with the exudates from the wound.

As is understood, by rapidly dissolving or gelling on contact with the exudates from the wound, the abovementioned web of microfibers or nanofibers will form an intermediate layer, which is not adhered to the wound, immediately (at most a few seconds) after the first deployment of the dressing, thus making it possible, if need be, to painlessly remove the dressing from the wound and to reposition it.

It has been found that, despite the presence of this web of water-soluble or gellable microfibers or nanofibers, the dressing retains the same adhesion profile on removal and the same absorption properties as a dressing having the same composition but not comprising these microfibers or nanofibers. In particular, there was not observed, with these novel dressings, migration of the exudates beyond or around the part of the surface of the adhesive mass covered with the layer formed after gelling or with dissolution of the web of microfibers or nanofibers, which might have detrimentally affected the adhesive properties or absorption and cohesion properties of the dressing by creating an absorption differential between these two areas.

According to a specific characteristic, this web can comprise particles of active substances which will be rapidly released and which will facilitate the healing of the blister or wound.

The dressing according to the present invention can be easily obtained by simple deposition of microfibers or nanofibers on the surface intended to come into contact with the wound of an adhesive dressing comprising hydrocolloids.

The adhesive masses comprising hydrocolloids capable of being used for the manufacture of the dressing in accordance with the invention are those normally used by a person skilled in the art for the manufacture of hydrocolloid adhesive dressings.

Generally, the adhesive masses of the dressings according to the invention exhibit an adhesive strength of at least 150 cN/cm and preferably of between 200 cN/cm and 8 N/cm, measured on a sheet of steel according to European standard EN 1939 (measurements at 90°, rate of 100 mm/min).

Generally, these adhesive masses will be composed of an elastomeric matrix comprising hydrocolloid particles and one (or more) compound(s) intended to confer adherence properties on said mass known under the name of “tackifier(s)”.

“Elastomeric matrix” is intended here to denote a composition comprising one (or more) elastomer(s) chosen from poly(styrene-olefin-styrene) block copolymers, the olefin blocks of which can be composed of isoprene, butadiene, ethylene-butylene or ethylene-propylene units and their mixtures.

In the context of the present invention, preference will be given to poly(styrene-isoprene-styrene) triblock copolymers (abbreviation: poly(SIS)) and to the blends of poly(SIS) triblock copolymers and poly(styrene-isoprene) diblock copolymers, and in particular to the poly(SIS) having a styrene content of between 14 and 52% by weight and preferably between 14 and 30% by weight, with respect to the weight of said poly(SIS).

Such products, which are well known to a person skilled in the art, are, for example, sold by Kraton under the Kraton® D name or by Dexco Polymers LP under the Vector® name.

Mention may in particular be made, among the preferred poly(SIS) triblock copolymers, of the products sold under the names Kraton® D-1111CS, Kraton® D-1107 or Kraton® 1161, Vector® 4114 and Vector® 4113.

Polystyrene-butadiene-styrene) triblock copolymers can also be used in the context of the invention.

Mention may in particular be made, among these poly(styrene-butadiene-styrene) copolymers, of the product sold under the name Kraton® D-1102 by Kraton.

Preferably, the elastomers forming the elastomeric matrix will be present, in the adhesive mass of the dressings according to the invention, in an amount of 10 to 30% by weight and preferably of 15 to 25% by weight, of the total weight of the adhesive mass.

Generally, the abovementioned elastomeric matrix incorporates one (or more) hydrocolloid(s).

“Hydrocolloid” is intended here to denote any compound normally used by a person skilled in the art for its ability to absorb hydrophilic liquids, such as water, physiological saline or exudates from a wound.

Mention may be made, as example of hydrocolloid capable of being used in the context of the invention, of pectin, alginates, natural vegetable gums, such as, in particular, karaya gum, cellulose derivatives, such as, in particular, carboxymethylcelluloses and their alkali metal salts, in particular sodium or calcium salts, and synthetic polymers based on acrylic acid salts, known under the name of “superabsorbents”, such as, in particular, the products sold by BASF under the name Luquasorb® 1003 or by CIBA Specialty Chemicals under the name Salcare® SC91. Of course, mixtures of these products can be used as hydrocolloids.

It should be noted that these compounds are present in the elastomeric mass and, although capable of gelling on contact with the exudates, their possible gelling can only be observed after a relatively long period of time, which can be as much as several hours, so that these compounds are not capable of solving the problem of the repositioning of the dressing.

The preferred hydrocolloids in the context of the present invention are the alkali metal salts of carboxymethylcellulose, in particular sodium carboxymethylcellulose.

The amount of hydrocolloid(s) incorporated in the elastomeric matrix will be adjusted as a function of the desired level of absorption. Generally, the amount of hydrocolloid(s) can be of the order of 2 to 50% by weight, with respect to the total weight of the adhesive mass.

In the context of the present invention, use will preferably be made of an amount of hydrocolloid(s) of between 20 and 50% by weight, with respect to the total weight of the adhesive mass, if it is desired to produce an absorbent dressing of the type of those described in the documents EP 1 061 965, EP 1 165 717 and EP 0 927 051.

The elastomeric matrix comprising hydrocolloid particles is rendered adhesive by the addition of “tackifying” products, such as those which are normally used by a person skilled in the art in the preparation of pressure-sensitive adhesives based on elastomers and in particular on poly(styrene-olefin-styrene) block copolymers. Reference may be made, for a detailed description of these products, to the documents of the state of the art mentioned above or to the work by Donatas Satas, “Handbook of Pressure Sensitive Technology”, 3rd edition, 1999, pages 346 to 398.

Generally, use may be made of one (or more) tackifying product(s) which will be incorporated in the hydrocolloid mass in a broad proportion of the order of 1 to 70% by weight, with respect to the total weight of the adhesive mass, which will be determined as a function of the nature and relative proportion of the other constituents of this mass, in order to obtain the adhesiveness desired for the final composition.

Preferably, the tackifying product(s) will represent from 10 to 40% by weight, of the total weight of the hydrocolloid mass.

The tackifying products capable of being used in the context of the present invention can be chosen from tackifying resins, polyisobutylenes of low molecular weight, polybutenes of low molecular weight or their mixtures.

Mention may be made, among the tackifying resins capable of being used according to the invention, of modified terpene or polyterpene resins, rosin resins, hydrocarbon resins, mixtures of cyclic, aromatic and aliphatic resins etc., or mixtures of these resins.

Such products are sold, for example:
by Goodyear under the Wingtack® name, such as, in particular, the synthetic resin formed of C₅/C₉ copolymers (Wingtack® 86) or the resin based on synthetic polyterpene (Wingtack® 10);
or by Hercules under the Kristalex® name, such as, in particular, the resin based on α-methylstyrene (Kristalex® 3085).

These tackifying resins can be used alone or as a mixture with other tackifiers, preferably in a proportion of 20 to 50% by weight and more particularly of 25 to 35% by weight, with respect to the total weight of the adhesive mass.

Mention may be made, among the polybutenes of low molecular weight capable of being used according to the invention, for example, of the products sold by the company BP Chimie under the Napvis® name, in particular the product sold under the name Napvis® 10.

These polybutenes can be used alone or as a mixture with other tackifiers, preferably in a proportion of 5 to 30% by weight and more particularly of 8 to 15% by weight, with respect to the total weight of the adhesive mass.

Mention may be made, among the polyisobutylenes of low molecular weight capable of being used according to the invention, of the polyisobutylenes having a molecular weight of the order of 40 000 to 80 000 daltons, such as, for example, the products sold by BASF under the Oppanol® name and in particular the products sold under the names Oppanol® B12 and Oppanol® B15.

These polyisobutylenes can be used or as a mixture with other tackifiers, preferably in a proportion of 5 to 30% by weight and more particularly 8 to 15% by weight, with respect to the total weight of the adhesive mass.

Various additional compounds can be added to the elastomeric matrix comprising the abovementioned tackifying and hydrocolloid compounds in order to obtain hydrocolloid adhesive masses which exhibit optimized properties of elasticity, adhesion, stability over time and cohesion.

Such compounds are, for example, stabilizers, such as, in particular, antioxidants, plasticizers, such as, in particular, polybutenes or plasticizing oils, or agents which make it possible to improve the cohesion, such as, in particular, butyl rubbers or polyisobutylenes of high molecular weight.

Mention may be made, among the polyisobutylenes of high molecular weight capable of being used according to the invention to improve the cohesion of the adhesive mass, of polyisobutylenes having a molecular weight of the order of 400 000 to 2 000 000 daltons, such as, for example, the products sold by BASF under the names Oppanol® B12 SFN or Oppanol® B30 SF.

These polyisobutylenes of high molecular weight can be used alone or as a mixture, preferably in a proportion of 2 to 20% by weight and more particularly of 5 to 15% by weight, with respect to the total weight of the adhesive mass.

“Stabilizer” is intended here to denote any compound capable of providing stability, with regard to oxygen (antioxidant), heat, ozone and ultraviolet radiation, to those compounds used in the formulation of the adhesive hydrocolloid masses, in particular tackifying resins and block copolymers. These stabilizing compounds are well known and can be used alone or as a mixture.

Mention may be made, among the antioxidizing compounds capable of being used according to the invention, of phenolic antioxidants, such as, for example, the products sold by Ciba-Geigy under the names Irganox® 1010, Irganox® 565 and Irganox® 1076, and sulfur-comprising antioxidants, such as, for example, zinc dibutyldithiocarbamate, sold by Akzo under the name Perkacit® ZDBC.

These compounds can be used alone or as a mixture, preferably in a proportion of 0 to 2% by weight and more particularly 0.1 to 0.6% by weight, with respect to the total weight of the hydrocolloid mass.

In the context of the present invention, use will preferably be made of the combination of Irganox® 1010 and Perkacit® ZDBC.

Mention may be made, among the plasticizing compounds capable of being used according to the invention, of the plasticizers normally used by a person skilled in the art in the preparation of hydrocolloid adhesive masses and in particular polybutenes, such as, for example, the products sold by BP Chemicals under the name Napvis® 10, plasticizing oils or phthalate derivatives, such as dioctyl phthalate.

The use of plasticizing oils is particularly preferred in the context of the present invention.

“Plasticizing oil” is intended here to denote the mineral or vegetable oils commonly employed by a person skilled in the art to plasticize the block copolymers of the styrene-olefin-styrene type used in the composition of hydrocolloid adhesive masses.

These mineral oils are generally composed of mixtures in variable proportions of compounds of paraffinic, naphthenic or aromatic nature.

Mention may be made, among the plasticizing oils capable of being used according to the invention, of the products sold by Shell under the Ondina® and Risella® names, which are composed of mixtures based on naphthenic and paraffinic compounds, or under the Catenex® name, which are composed of mixtures based on naphthenic, aromatic and paraffinic compounds.

In the context of the present invention, use will preferably be made of the mineral plasticizing oil sold under the name Ondina® 68.

These plasticizing compounds can be used alone or as a mixture, preferably in a proportion of 5 to 20% by weight and more particularly of 7 to 15% by weight, with respect to the total weight of the hydrocolloid mass.

The hydrocolloid adhesive mass of the dressings according to the invention can also comprise one or more surface-active compound(s) in an amount of less than or equal to 10% by weight, preferably of less than or equal to 5% by weight, with respect to the total weight of the hydrocolloid mass.

A preferred surface-active compound in the context of the present invention is the compound sold under the AcResin® name.

Another preferred surface-active compound in the context of the present invention is polysorbate 80, such as, for example, the product sold by Seppic under the name Montanox® 80.

In the context of the present description, the expression “web of microfibers or nanofibers” is intended to denote an assembly of microfibers preferably having a diameter of between 1 and 50 μm, more preferably between 1 and 25 μm, and/or of nanofibers having a diameter of between 20 and 1000 nm, more preferably of between 50 and 500 nm, which are positioned, preferably according to a monolayer arrangement, while forming a web analogous to a nonwoven, that is to say a random assembly of microfibers and/or nanofibers which is held in the form of a sheet by friction, cohesion or adhesion.

The webs of microfibers or nanofibers according to the invention can be obtained by an aerodynamic drawing of molten polymers or of polymers in aqueous or solvent solution according to the technology known under the name of centrifugal spinning.

The webs of microfibers or nanofibers can also be obtained by the process known under the term of electrospinning.

Electrospinning is a technology which makes possible the preparation of nanofibers by evaporation of a polymer solution or dispersion in a high voltage electric field. More specifically, this process consists in subjecting a sufficiently fluid solution or dispersion of a material, exiting from a very thin nozzle, to an electrical potential of the order of 5 to 50 kV. Under the effect of this electric field, the drop exiting from the nozzle becomes electrically charged, assuming a substantially conical shape, and can be drawn into a jet to form a very thin fiber having a size from the nanometric to micrometric scale when the voltage is sufficiently high to break the surface tension of the drop. The fibers thus formed can be collected and stored in the state, for example on a collector, or else deposited on a backing in random fashion, to form a web similar to a nonwoven. This technology is particularly suitable for the preparation of microfibers or nanofibers based on polyvinyl alcohol.

Whatever the technology used, the web of microfibers or nanofibers can be formed beforehand and then deposited on the surface of the hydrocolloid adhesive mass. Alternatively, the web can be formed “in situ” by using the surface of the hydrocolloid adhesive mass as backing during the formation of the web.

As is understood, the microfibers or nanofibers used in the context of the present invention are known products.

Generally, the microfibers or nanofibers forming the web are composed, for 90% at least and preferably for 95% at least of them, of one or more materials chosen from natural or synthetic polymers, and capable of dissolving or gelling in less than 10 seconds and more preferably in less than 1 second on contact with the exudates from the wound.

The expression “material capable of dissolving” is intended to denote any material capable of dissolving in the exudates from the wound with the formation of a homogeneous solution.

The expression “material capable of gelling” is intended to denote any material capable of forming a gel on contact with the exudates from the wound, i.e. a substance exhibiting a viscosity greater than that of water, for example greater than 10⁻³ Pa·s and preferably greater than 10⁻² Pa·s.

Mention may be made, among the materials capable of being used according to the invention, of water-soluble or water-dispersible polymers and in particular:

polyvinyl alcohol (PVA);

poly(vinylpyrrolidone) (PVP);

polyethyleneimine (PEI);

polyethylene oxides (PEOs);

carboxymethylcellulose,

alginates;

and the mixtures of these compounds.

Excellent results have been obtained using microfibers or nanofibers of polyvinyl alcohol, which thus constitutes a preferred material for the implementation of the invention.

The microfibers or nanofibers used in the context of the present invention can be different in nature, depending on the degree of solubility desired, the mechanical strength desired or the temperature stability desired.

According to a specific embodiment of the invention, the web of microfibers or nanofibers comprises one (or more) active substance(s).

The active substances capable of being used in the context of the present invention can be chosen from antifungals, antimicrobials or antibacterials, such as silver sulfadiazine, pH regulators, healing accelerators, such as hyaluronic acid, vitamins, moisturizing agents, trace elements, local anesthetics, odor trappers, menthol, methyl salicylate, hormones, anti-inflammatories and the mixtures of these compounds.

A preferred active substance in the context of the invention is hyaluronic acid.

The active substances can be incorporated in the web, either before or after the latter has been formed at the surface of the hydrocolloid adhesive mass, by any means which makes possible the deposition thereof. Alternatively, these active substances can be incorporated in the microfibers or nanofibers before the latter are deposited in the form of a web at the surface of the hydrocolloid adhesive mass.

In the case where the web of microfibers or nanofibers is formed by electrospinning, the active substances can be incorporated in the solution or dispersion which is subjected to the electric field (for example in a polyvinyl alcohol solution, in the case of microfibers or nanofibers of polyvinyl alcohol), thus making it possible to incorporate these active substances directly in the web of microfibers or nanofibers.

According to the invention, the microfibers or nanofibers are positioned so as to form a web at the surface of the hydrocolloid adhesive mass intended to come into contact with the wound. In other words, these microfibers or nanofibers will not be present inside the hydrocolloid adhesive mass.

Generally, the web formed from these microfibers or nanofibers will cover between 5 and 90% and preferably between 10 and 60% of the surface of the adhesive mass coming into contact with the wound, according to the application envisaged. Thus, the degree of covering of the surface of the adhesive mass coming into contact with the wound will be of the order of 10 to 50% for a dressing intended for the treatment of blisters and of the order of 20 to 70% for a dressing intended for the treatment of bedsores or ulcers.

The microfibers or nanofibers can be incorporated in the dressing in accordance with invention by any process which makes possible their deposition at the surface of the hydrocolloid adhesive mass in the form of a web.

For example, this operation can advantageously be carried out by simple deposition of a web formed beforehand or by the use of the electrospinning or centrifugal spinning processes described above.

According to a preferred embodiment of the invention, the microfibers or nanofibers will be positioned solely on the area of the dressing intended to come into contact with the area of the wound to be treated.

For example, the microfibers or nanofibers can be deposited on the areas intended to constitute the central part of the dressings.

The microfibers or nanofibers can be deposited according to any geometrical shape, for example a square, rectangular or oval shape, which may or may not be openwork. The amount of microfibers and nanofibers deposited can vary within wide proportions and will generally be between 1 and 100 g and preferably between 1 and 30 g per square meter of adhesive mass.

The dressings of the invention can be of different types but will preferably comprise a backing.

The adhesive mass carrying the web of microfibers or nanofibers can be complexed to a backing of the type of those used in the dressings sold by Laboratoires Urgo under the Algoplaque® names or by Convatec under the Duoderm® name or also by Coloplast under the Comfeel® name. Such dressings are described in the documents FR 2 392 076, FR 2 495 473, WO 98/10801 and EP 264 299.

Generally, the choice of the backing will be made as a function of the properties required (leaktightness, elasticity, and the like) in the desired application.

Thus, the dressing according to the invention can comprise a backing, such as a film formed of one or more layers and with a thickness which can vary from 5 to 150 μm, a nonwoven or a foam having a thickness of 10 to 500 μm, on to which the hydrocolloid adhesive mass has been coated in a continuous or noncontinuous fashion.

These backings based on synthetic or natural materials are well known to a person skilled in the art.

Mention may thus be made, among the backings in the form of a foam which can be used in the context of the invention, of polyethylene, polyurethane or PVC foams, for example.

Mention may be made, among the nonwoven backings which can be used in the context of the invention, of nonwovens made of polypropylene, polyethylene, polyurethane, polyamide, or polyester, for example.

In the context of the present invention, preference will be given to the use of backings in the form of films, in particular polyurethane films, such as, for example, the films sold by Smith and Nephew under the Lasso® reference which are produced from polyurethane sold by BF Goodrich under the Estane® name; low-density polyethylene films, such as, for example, the films sold by Sopal; films based on thermoplastic polyether/polyester copolymer, such as, for example, products sold by Dupont de Nemours under the Hytrel® name; or complex films combining a polyurethane film and a nonwoven.

According to an alternative embodiment of the invention, the dressing can comprise an absorbent layer positioned between the backing and the adhesive hydrocolloid mass. This absorbent layer can be composed of any type of absorbent material, such as, for example, a foam (such as, in particular, a polyurethane foam), a nonwoven, a superabsorbent polymer layer or a combination of these materials.

The dressings produced from the hydrocolloid adhesive mass according to the invention can be provided in any geometrical shape, for example square, rectangular, circular or oval. Likewise, they can have any size and the latter will be adapted as a function of the surface of the part to be treated or protected. For example, a dressing intended for the treatment of blisters will be provided in a rectangular form with a length of approximately 7 cm and a width of approximately 4 cm, while a dressing intended for the treatment of ulcers will be provided in a square form with a side length of 10 cm.

In practice, the surface of the hydrocolloid adhesive mass carrying the web of microfibers or nanofibers can be covered with a protective film intended to be removed, for example by peeling; before the deployment of the dressing on the wound and/or on the skin.

The assembly thus formed can itself be wrapped in a leaktight protection formed, for example by means of polyethylene/aluminum complexes or in blister packs.

The invention will be illustrated by the following nonlimiting examples.

### EXAMPLE 1

### Preparation of a Standard Hydrocolloid Dressing

A hydrocolloid adhesive mass was prepared composed of the following compounds (amount expressed by weight per 100 grams of mass).

| | | Amount (by |
|---|---|---|
| | | weight per |
| No. | Compound | 100 grams) |
| 1 | Elastomer | 14.144 |
| | Styrene-Isoprene-Styrene/Styrene-Isoprene | |
| | block copolymer (sold under the name Vector ® | |
| | 4114A by Dexco Polymers LP) | |
| 2 | Cohesion-improving agent | 3.536 |
| | Polyisobutene (sold under the name Oppanol ® | |
| | B12SFN by BASF) | |
| 3 | Plasticizer | 12.376 |
| | Mineral oil (sold under the name Ondina ® 933 | |
| | by Shell) | |
| 4 | Surfactant | 6.542 |
| | UV-crosslinkable acrylic ester copolymer (sold | |
| | under the name AcResin ® A258UV by BASF) | |
| 5 | Tackifying resin | 26.521 |
| | Synthetic hydrocarbon resin (sold under the | |
| | name Wingtack ® 86 by Safic Alcan) | |
| 6 | Hydrocolloid | 35.714 |
| | Carboxymethylcellulose (sold under the name | |
| | CMC Blanose ® 7H4XF by Hercules) | |
| 7 | Surfactant | 0.460 |
| | Polysorbate 80 (sold under the name | |
| | Montanox ® 80 by Seppic) | |
| 8 | Antioxidant | 0.354 |
| | Zinc dibutyldithiocarbamate (sold under the | |
| | name Perkacit ® ZDBC by Flexsys (distributor | |
| | Arnaud)) | |
| 9 | Antioxidant | 0.354 |
| | Pentaerythritol tetrakis(3-(3,5-di(tert-butyl)-4- | |
| | hydroxyphenyl)propionate) (sold under the | |
| | name Irganox ® 1010 by Ciba Speciality | |
| | Chemicals) | |

This hydrocolloid adhesive mass was prepared by carrying out the following process:

Compounds 1, 2, 3, 8 and 9 were introduced into a Z-arm blender at a set temperature of 140° C.

Compound 4 was added at the 30th minute.

Compound 7 was added at the 40th minute.

Compound 5 was added at the 45th minute.

Compound 6 was added at the 60th minute.

The blender was emptied at the 80th minute.

The adhesive hydrocolloid mass was heated to a temperature of the order of 110 to 130° C. and then coated on to a backing composed of a polyurethane film having a thickness of 30 μm. The complex formed of the hydrocolloid mass and backing was subsequently cut into individual dressings covered, on the free adhesive hydrocolloid surface, with protective tabs made of silicone polyester.

### EXAMPLE 2

### Preparation of a Dressing According to the Invention

The adhesive hydrocolloid mass prepared in example 1 was heated to a temperature of the order of 110 to 130° C. and then coated on to a backing composed of a polyurethane film having a thickness of 30 μm. The complex formed by the hydrocolloid adhesive mass and the polyurethane film was cut into individual dressings.

A web formed beforehand by electrospinning nanofibers based on polyvinyl alcohol (product sold by Seppic under the name Mowiol 40-88) having a diameter of 100 to 200 nm was subsequently deposited on the free surface (not bonded to the backing) of the hydrocolloid adhesive mass.

These nanofibers were deposited at the center of the dressing by simple depositing of the web of nanofibers in an amount of between 10 and 15 g/m².

The surface of the adhesive hydrocolloid mass carrying the nanofibers was covered with protective tabs made of silicone polyester.

### EXAMPLE 3

### Demonstration of the Properties of the Dressing in Accordance with the Invention

The adhesive strength of the dressings as prepared in examples 1 and 2, after contact with physiological saline to simulate the exudation from a wound, was measured in order to evaluate the difference in behavior on removal between a dressing comprising a conventional hydrocolloid adhesive mass (example 1) and a dressing according to the invention comprising a hydrocolloid adhesive mass, the surface of which intended to come into contact with the skin carries a web of nanofibers (example 2).

To this end, use was made of the “probe tack” method, which is targeted at measuring the detachment of a cylindrical probe applied to an adhesive with a pressure stress P₀ for a given time t under predetermined temperature and humidity conditions.

Equipment

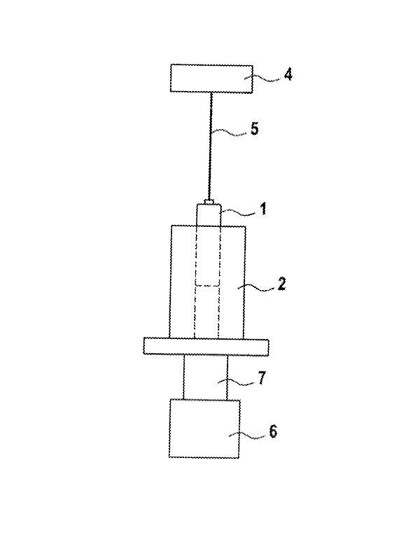

More specifically, this method was carried out using the device represented in FIG. 1.

This device is essentially composed:
of a cylindrical probe 1 exhibiting a finely polished end capable of moving longitudinally (from the bottom upwards in the example represented) through a probe guide 2 composed of a cylinder pierced in its center with a free hole which makes possible the free frictionless passage of the probe;
of an electronic dynamometer comprising a movable gripping element 4 which can be connected to the probe 1 via a small chain 5 and a base 6 on which a plate 7 can be fitted, said dynamometer being in addition connected to a system for acquiring and recording data, such as in particular the rate of descent of the probe, the contacting pressure of the probe on the adhesive, the contact time between the probe and adhesive and the rate of rise of the probe.

Samples

### Standard Dressing

A substantially square dressing with a side length of 20 cm was prepared by following the experimental protocol of example 1.

This dressing was covered with a double-sided adhesive tape on the face forming the backing. A pressure roll was employed in order to cause said tape to adhere well to the surface of the dressing.

Circular samples with the same surface area as the probe were cut from the assembly thus formed by the dressing and the abovementioned adhesive tape using a circular hollow punch with a diameter of 30 mm.

### Dressing According to the Invention

A second set of samples of the same nature and of the same size was prepared.

Nanofibers based on polyvinyl alcohol, such as those used in example 2, were deposited in the form of a web on the surface of these samples.

Procedure:

The plate 7 was fitted to the base 6 of the dynamometer.

The small chain 5 of the probe was attached to the movable gripping element 4 of the dynamometer.

The probe 1 was cleaned with a solvent (this operation being repeated before each measurement).

A sample (double-sided side) as prepared above was applied to the probe.

50 μl of physiological saline were deposited on the plate 7 in the middle of the area delimited by the probe guide.

The parameters of the dynamometer (rate of descent, contacting pressure, contact time and rate of rise) were programmed.

The measurement was carried out by performing the following operations: descent of the probe, bringing the adhesive sample and the physiological saline into contact.

The detachment force was recorded in kPa and was translated into tear-off stress S according to the relationship:

S=F/A

where F=Force expressed in N and A=Area expressed in m².

Parameters of the Test:

Application stress: 1.7 kPa (aluminum probe with a weight of 121 g and a diameter of 30 mm)
Test rate: 300 mm/min
Pressure roll: 4 kg
Contact time: 1 s
The measurements were carried out on 3 samples of each of the standard dressing and dressing according to the invention and repeated 6 times.

Results:

The results of the measurements thus carried out, expressed in kPa, have been given in table I.

TABLE I

| Test No. | Example 1 | Example 2 |
|---|---|---|
| 1 | 138.8 | 0.2 |
| 2 | 182.8 | 0.3 |
| 3 | 171.2 | 0.6 |
| 4 | 160.7 | 0.6 |
| 5 | 134.4 | 0.9 |
| 6 | 194 | 0.7 |

### Observations of the Behavior of the Dressings According to Examples 1 and 2 in the Presence of Physiological Saline

| | Observations after contact with |
|---|---|
| Conditions | physiological saline |
| Example 1 + 50 μl of | Complete gelling after 15 minutes |
| physiological saline | |
| Example 2 + 50 μl of | Dissolution in one second |
| physiological saline | |

### Conclusions

The virtually complete dissolution of the web is very fast (1 s).

The “probe tack” test has made it possible to state that the adhesive strength on removal of the hydrocolloid adhesive dressing according to the invention is much lower than that of a standard webless dressing.

When the hydrocolloid adhesive mass of the dressing is covered with a web of microfibers or nanofibers, the adhesive strength on removal of the dressing becomes minimal, indeed even virtually zero, when it comes into contact with physiological saline, even in a very small amount.

Thus, a dressing according to the invention, that is to say comprising a hydrocolloid adhesive mass, the surface of which is intended to come into contact with the wound is covered, at least in part, with water-soluble or gellable microfibers or nanofibers, can be easily repositioned after a first application, this being the case in the seconds which follow said first application.

1. A wound dressing comprising an adhesive mass comprising hydrocolloids, wherein the dressing comprises, on a portion at least of the surface of the adhesive mass intended to come into contact with the wound in the position of use, a web of microfibers or nanofibers which are composed, for 90% at least and preferably 95% at least of them, of one (or more) material(s) chosen from natural or synthetic polymers, and capable of dissolving or gelling in less than 10 seconds and more preferably in less than 1 second on contact with the exudates from the wound.

2. The wound dressing as claimed in claim 1, wherein the abovementioned microfibers have a diameter of between 1 and 50 μm, preferably between 1 and 25 μm.

3. The wound dressing as claimed in claim 1, wherein the abovementioned nanofibers have a diameter of between 20 and 1000 nm, preferably between 50 and 500 nm.

4. The wound dressing as claimed in claim 1, wherein the abovementioned microfibers or nanofibers are present in an amount of 1 to 100 g, preferably of 1 to 30 g, per m² of surface area of adhesive mass.

5. The wound dressing as claimed in claim 1, wherein the abovementioned web of microfibers or nanofibers covers from 10 to 60% of the surface of the adhesive mass intended to come into contact with the wound in the position of use.

6. The wound dressing as claimed in claim 1, wherein the abovementioned main material constituting the microfibers or nanofibers is chosen from water-soluble or water-dispersible polymers and in particular:
polyvinyl alcohol (PVA);
poly(vinylpyrrolidone) (PVP);
polyethyleneimine (PEI);
polyethylene oxides (PEOs);
carboxymethylcellulose,
alginates;
and the mixtures of these compounds.

7. The wound dressing as claimed in claim 1, wherein the abovementioned material constituting the microfibers or nanofibers is polyvinyl alcohol.

8. The wound dressing as claimed claim 1, wherein the web of microfibers or nanofibers comprises one or more active substance(s) chosen from healing accelerators, antifungals, antimicrobials or antibacterials, moisturizing agents, local anesthetics, anti-inflammatories or a mixture of these compounds.

9. The wound dressing as claimed in claim 8, wherein the abovementioned active substance is hyaluronic acid.

10. The wound dressing as claimed in claim 1, wherein it comprises a backing.

11. The wound dressing as claimed in claim 1, wherein it comprises an absorbent layer situated between the backing and the hydrocolloid mass.
